(19) United States
(12) Patent Application Publication
(10) Pub. No.: US 2012/0065619 A1
(43) Pub. Date: Mar. 15, 2012

(54) METHOD FOR LABIAL, VAGINAL, TRANSMUCOSAL AND TRANSDERMAL METERED DOSE DISPENSING

(76) Inventor: Marla Ahlgrimm, Madison, WI (US)

(21) Appl. No.: 13/232,799

(22) Filed: Sep. 14, 2011

Related U.S. Application Data
(60) Provisional application No. 61/382,571, filed on Sep. 14, 2010.

(51) Int. Cl. A61M 31/00; A61M 35/00

(52) U.S. Cl. 604/515; 604/290; 604/310; 604/187

(57) ABSTRACT

A method for using a dispensing apparatus having a fluid insert with an airless channel configured for labial, vaginal, transmucosal, and transdermal delivery of a therapeutic substance is disclosed.

### CROSS REFERENCE TO RELATED APPLICATIONS

This is a non-provisional patent application and claims priority to U.S. Provisional Patent Application Ser. No. 61/382,571, filed on Sep. 14, 2010, which is herein incorporated by reference in its entirety.

### FIELD

The present disclosure relates to a method for administration of a therapeutic substance, and in particular to a method for labial, vaginal, transmucosal, and transdermal metered dose dispensing of the therapeutic substance.

### BACKGROUND

Conventional routes of drug delivery are invariably restricted to oral delivery for systemic action and topical for local action. Other routes of self-administration such as inhalation, suppository and injections, are also used for delivery of therapeutic substances. In the mid 1980s, subdermal and intrauterine routes became familiar for sustained medication, since such routes required only a single intervention by the doctor. Transdermal and intranasal routes developed later offered alternatives for self-administration, while non-oral routes were developed over the years since such routes avoided the hepatic metabolism.

Wider choices of sites for drug administration are currently being explored, the vagina being one of them. Non-invasive transmucosal and vaginal routes offer many advantages like avoiding first-pass effect, self and/or single administration that is effective for weeks or even months, and providing optimum pharmacokinetic profiles. Unfortunately, many misperceptions and poor knowledge about this route has led to a social stigma, leading to reluctance to use vaginal medications.

The female genitalia includes the interlabial space, located between the inside surfaces of the labia majora and extending past the vestibule of the vagina and includes the labia minora, clitoris, urethral orifice, vaginal orifice and the vagina. It has been found that that local and transmucosal administration of many new and existing therapeutic agents can be facilitated by application of drug to the labial tissue or within the vagina. However, because of the sensitive anatomy of the perineum and labial tissue and vagina, it has been difficult to administer therapeutic agents in this fashion. Although creams and gels can be applied to the labial area and to the vagina through the use of creams, suppositories, or tablets, administration is often messy, inconvenient or difficult to administer, may be the cause of inaccurate dosing due to measuring errors, a cause of drug contamination and degradation, and the source of drug being transferred to others from fingers, tubes, and independent applicators. Therefore, there is a need in the art for a method of administration of drugs by vaginal, transmucosal and transdermal routes using a method that allows for airless, metered vaginal, transmucosal, or transdermal drug delivery that is convenient, painless and avoids the first pass metabolism for both local and systematic drug therapy.

### SUMMARY

In one embodiment, a method of administration may include:
providing a dispensing apparatus comprising:
   an outer casing defining a distal end having an applicator forming a central aperture and a proximal end forming a first opening in communication with a first channel defined along a length of the outer casing;
   a fluid insert disposed with the channel, the fluid insert defining a distal end in fluid flow communication with the central aperture and a proximal end defining a second opening in communication with a second channel configured to store a predetermined amount of fluid material therein; and
   a sealing plug configured to engage the second opening and seal the second channel;
actuating the dispensing apparatus;
dispensing the fluid material through the applicator for labial or vaginal delivery.

In another embodiment, a method of administration may include:
providing a dispensing apparatus comprising:
   an outer casing defining a distal end having an applicator forming a central aperture and a proximal end forming a first opening in communication with a first channel defined along a length of the outer casing;
   a fluid insert disposed with the channel, the fluid insert defining a distal end in fluid flow communication with the central aperture and a proximal end defining a second opening in communication with a second channel configured to store a predetermined amount of fluid material therein; and
   a sealing plug configured to engage the second opening and seal the second channel;
actuating the dispensing apparatus;
dispensing the fluid material through the applicator for transmucosal delivery.

In one embodiment, a method of administration may include:
providing a dispensing apparatus comprising:
   an outer casing defining a distal end having an applicator forming a central aperture and a proximal end forming a first opening in communication with a first channel defined along a length of the outer casing;
   a fluid insert disposed with the channel, the fluid insert defining a distal end in fluid flow communication with the central aperture and a proximal end defining a second opening in communication with a second channel configured to store a predetermined amount of fluid material therein; and
   a sealing plug configured to engage the second opening and seal the second channel;
actuating the dispensing apparatus; and
dispensing the fluid material through the applicator for transermal delivery to an individual.

In yet another embodiment, a dispensing apparatus for administration of a fluid material may include:
an outer casing defining a distal end having an applicator forming a central aperture and a proximal end forming a first opening in communication with a first channel defined along a length of the outer casing;
a fluid insert disposed with the channel, the fluid insert defining a distal end in fluid flow communication with the central aperture and a proximal end defining a second opening in communication with a second channel configured to store a predetermined amount of fluid material therein, wherein the channel is airless; and
a sealing plug configured to engage the second opening and seal the second channel.

### BRIEF DESCRIPTION OF THE DRAWINGS

FIG. 1 is a perspective view of one embodiment of a dispensing apparatus used with the method of administration;

FIG. 2 is an exploded view of the dispensing apparatus of FIG. 1 illustrating the various components of the dispensing apparatus;

FIG. 3 is a side view of the outer casing for the dispensing apparatus;

FIG. 4 is a cross-sectional view of the fluid insert for the dispensing apparatus showing the pump engaged to the fluid insert body;

FIG. 5 is a side view of the fluid insert illustrating a disassembled view of the pump and the plug insert relative engaged to the fluid insert body; and

FIG. 6 is a flow chart illustrating one method for using the dispensing apparatus in the method of administration.

Corresponding reference numbers indicate corresponding elements among the various view of the drawing. The heading used in the figures should not be interpreted to limit the scope of the claims.

### DETAILED DESCRIPTION

A method of administration using a dispensing apparatus for labial, vaginal, transmucosal, and transdermal drug delivery for local or systematic drug absorption is described herein. The method of administration also allows for targeted delivery of low lipid permeable active agents as well as the delivery of systemic pharmaceutical agents.

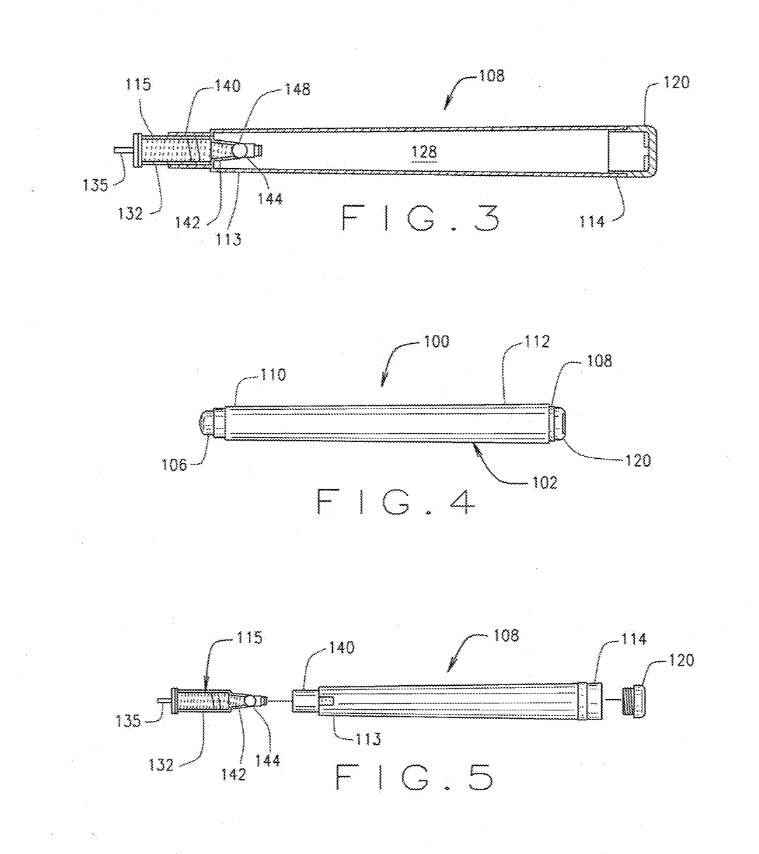

One embodiment of a dispensing apparatus used for the method of administration capable of transmucosal, interlabial, perilabial, vaginal, and transdermal drug delivery is generally indicated as 100 in FIGS. 1-5. In general, the dispensing apparatus 100 may include a fluid insert 108 for storage of a fluid or the like with the fluid insert 108 being disposed within a hollow elongated outer casing 102. The outer casing 102 forms a distal end 110 configured to engage a cap 104 and a proximal end 112 that defines an opening 130 in communication with a channel 126 (shown in phantom) formed along the interior of the hollow outer casing 102. In addition, the fluid insert 108 includes an elongated outer body 116 having a distal end 113 and a proximal end 114 with the outer body 116 defining an interior chamber 128 (shown in phantom) configured to store a predetermined amount of a material therein. In some embodiments, the material may be a liquid material, a gel material, a paste material, a semi-liquid material, a semi-solid material, and combinations thereof. In some embodiments, the material is capable of providing a delivery medium capable of drug delivery. The proximal end 114 of the fluid insert 108 is configured to engage a seal plug 120 for sealing off the proximal end 114 of the fluid insert 108 during assembly of the dispensing apparatus 100. In one embodiment, the seal plug 120 may be configured for snap fit engagement with the fluid insert 108, while in some embodiments an adhesive or other similar bonding material may be used to further secure the seal plug 120 with the fluid insert 108, if desired. In some embodiments, the interior chamber 128 may form an airless compartment that is completely filled with the fluid material, while in other embodiments air may fill a portion of the interior chamber 128 not filled with the fluid material.

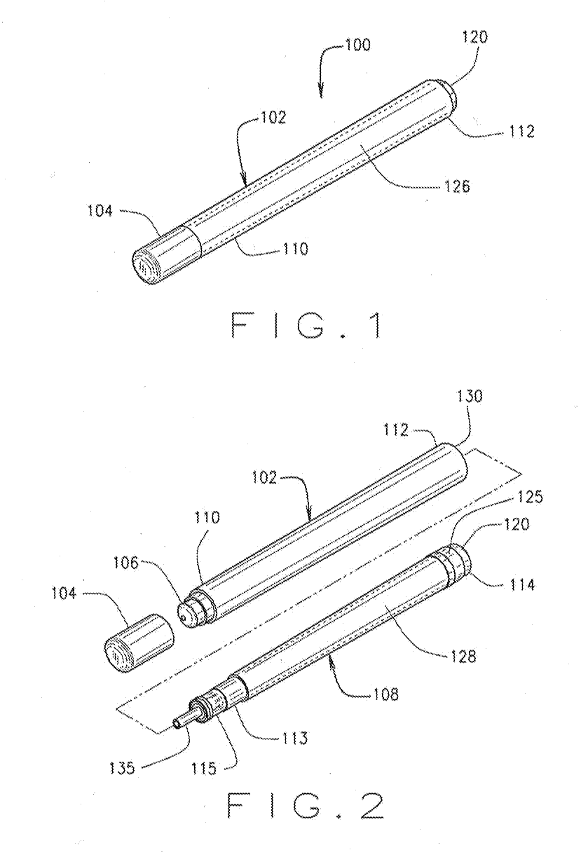

Referring to FIG. 2, an applicator 106 extends outwardly from the distal end 110 of the outer casing 102 and defines a generally rounded end 122 that defines a central aperture 124. The channel 126 of the outer casing 102 is configured to receive the fluid insert 108 therein as shall be described in greater detail below.

As further shown, the distal end 113 of the fluid insert 108 includes a pump 115 for dispensing the fluid material stored inside the interior chamber 128. In some embodiments, the pump 115 may be a standard pump, such as an EMSAR pump, PAV (A45) series having a 130 micro liter output capable of dispensing the fluid material with an internal check valve 148, such as an internal ball check valve arrangement, for regulating the amount of air permitted to enter the internal chamber 128 of the fluid insert 108. In some embodiments, the pump 115 is capable of pumping fluid material having a fluid viscosity ranging from at least 1000 cps to 10,000 cps, although other viscosities of lower or higher viscosity may also than the above fluid viscosity range may be considered. Referring to FIG. 4, the pump 115 includes a hollow tip portion 135 that extends outwardly from a pump body 132 and through the central aperture 124 of the applicator 106 when the fluid insert 108 is engaged to the outer casing 102. In some embodiments, the pump body 132 forms a main portion 140, an intermediate portion 142 and an intake portion 144, respectively, that each define progressively tapered configurations in decreasing size and length. The distal end 113 of the fluid insert 108 is configured to engage the pump 115 such that the intake portion 144 is disposed within the internal chamber 128 of the fluid insert 108 and in direct communication with a predetermined amount of fluid material stored therein, while the hollow tip portion 135 of the pump 115 and is in fluid flow communication with the central aperture 124 of the applicator 106. As such, the hollow tip portion 135 of the pump 115 is in fluid flow communication with the fluid material stored in the internal chamber 128 for dispensing the fluid material through the hollow tip portion 135 when the pump is activated.

During assembly of the dispensing apparatus 100, the fluid insert 108 may first be assembled such that the pump 115 is connected to the distal end 113 of the fluid insert 108 and the seal plug 120 is engaged to the proximal end 114 of the fluid insert 108 such that a predetermined amount of fluid material is stored inside the interior chamber 128. The assembled fluid insert 115 is then inserted into the channel 126 of the outer casing 102 such that the hollow tip portion 135 of the pump 115 is in fluid flow communication with the central aperture 130 of the applicator 106.

To dispense the fluid material from the dispensing apparatus 100, an external force is applied to the seal plug 120 of the fluid insert 108 which forces the fluid insert to move a short distance within the outer casing 102. The moving the fluid insert 108 forces retraction of a spring (not shown) into the pump 115 such that the hollow tip portion 135 is able to retract. When the hollow tip portion 135 is retracted into the pump body 132, fluid material is withdrawn from interior chamber 128 and then expelled through the central opening 130 of the applicator 106 by action of the released spring. In some embodiments, the dispensing apparatus 100 may include a spray pump (not shown) attached to a pump line that is in fluid flow communication with the fluid material stored in the fluid insert 108 that is actuated by applying and releasing an external force against the fluid insert 108 that forces the fluid material through the spray pump. In some embodiments, the dispensing apparatus 100 may be modified to include a plunger (not shown) which uses a plunging action to dispense the fluid material through the applicator 106. In some embodiments, a retaining ring 125 may be formed or slipper around the circumference of the fluid insert 108 adjacent or proximate the proximal end 114 and configured to interact with a structural arrangement that actuates the pump 115 to dispense a predetermined amount of fluid material when the dispensing apparatus is actuated. In some embodiments, the dispensing apparatus 100 may dispense a predetermined amount of fluid material in single or multiple doses. In those embodiments of the dispensing apparatus 100 that expel in multiple doses, each individual dose may constitute a different type of fluid material stored in separate compartments in the fluid insert 108 that effectively segregate each fluid material from the other fluid material to prevent cross contamination. In some embodiments, the same amount of fluid material is dispensed, while in other embodiments a different amount of fluid material is dispensed with each dose which can be adjusted with each dose.

In some embodiments, the dispensing apparatus 100 includes a meter actuating surface having multiple buttons, with each button dispensing a different amount of fluid material for a particular dose. In other embodiments, the meter actuating surface is a level, which allows measuring the fluid material in a finer range. In one embodiment, the meter actuating system may be an LED display, which displays the numbers and/or text containing information related to the number of doses applied, number of doses remaining, the required amount of fluid material for each particular dose, and the actual measurement of the fluid material for each dose to be dispensed from the dispensing apparatus 100.

In some embodiments, the dispensing apparatus 100 includes sound recording and replaying components to convey information; computer and communication components for wirelessly communicating information to the dispensing apparatus 100, such as instruction regarding the number doses and the predetermined amount of fluid material required for each dose.

In one aspect, the applicator 106 may be disposable and adapted for one-time use, while other applicators 106 may be sterilized and used more than once. In some embodiments, the applicator 106 may be integral and in other embodiments the applicator 106 may be disposable and configured to be disengaged from the outer casing 102. In those embodiments of the applicator 106 that are disposable, each applicator 106 may have a different size in either volume or shape configured to be engaged to the outer casing 102. In addition, each disposable-type applicator 106 may be configured for a snap-fit engagement with the outer casing or a mechanical trigger arrangement may be used to release the applicator 106 from the outer casing 102. For example, the applicator 106 may have a pointed tip, a blunt tip, a dome-shaped tip, a brush tip, a pad configuration, and a sponge configuration.

In some embodiments, the applicator 106 may be made from a material, which can be heated or chilled to increase the comfort level of an individual. Other applications may include controlling the fluidity or potency of the fluid material by changing the temperature of the fluid material inside the fluid insert 108 prior to dispensing each dose through the applicator 106.

In a first aspect, the dispensing apparatus 100 may be used for transmucosal delivery of a fluid material, such as a therapeutic substance including, but not limited to, pharmaceuticals, hormones, systemically active therapeutics, vaccines, large molecules, small molecules and nanoparticles. The method of administration using the dispensing apparatus 100 for labial, vaginal, transmucosal, and transdermal delivery of the fluid material avoids the disadvantages of oral intake of therapeutic substances, such as negative effects to the gastrointestinal system, liver and kidneys. This manner of administration also decreases the known fluctuations in absorption rates and the consistency of therapeutic levels commonly related to oral administration routes.

In a second aspect, the dispensing apparatus 100 may be used for transdermal delivery of a fluid material. The method of administration using transdermal delivery is preferable to topical administration since it provides for local delivery of systemic therapeutics similar to transmucosal delivery.

In a third aspect, the dispensing apparatus 100 may be used for vaginal or labial delivery of fluid material, such as a therapeutic substance including, but not limited to, hormones for hormone replacement therapy and other applications.

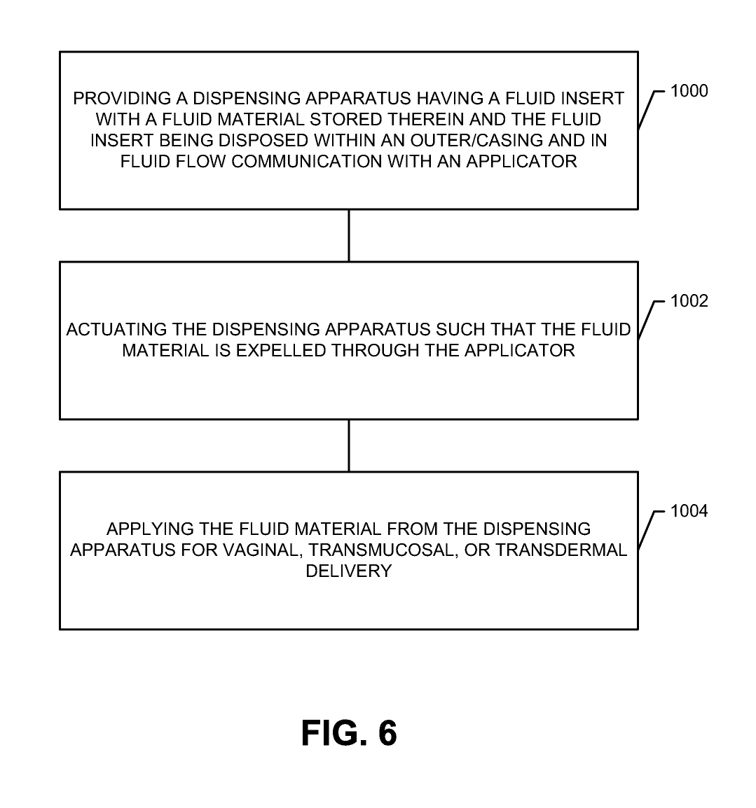

Referring to FIG. 6, one method of administration using the dispensing apparatus 100 is illustrated. At block 1000, the dispensing apparatus 100 is provided having a fluid insert 108 with a fluid material stored therein and the fluid insert 108 being disposed within an outer casing 102 and in fluid flow communication with an applicator 106. At block 1002, the dispensing apparatus 100 is actuated such that the fluid material is expelled through the applicator 106. At block 1004, the fluid material is applied from the dispensing apparatus 100 for labial, vaginal, transmucosal, and transdermal delivery.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A method of administration comprising:
providing a dispensing apparatus comprising:
an outer casing defining a distal end having an applicator forming a central aperture and a proximal end forming a first opening in communication with a first channel defined along a length of the outer casing;
a fluid insert disposed with the channel, the fluid insert defining a distal end in fluid flow communication with the central aperture and a proximal end defining a second opening in communication with a second channel configured to store a predetermined amount of fluid material therein; and
a sealing plug configured to engage the second opening and seal the second channel;
actuating the dispensing apparatus;
dispensing the fluid material through the applicator for labial or vaginal delivery.

2. The method of administration of claim 1, further comprising a pump disposed within the distal end of the fluid insert for expelling the fluid material through the central aperture.

3. The method of administration of claim 2, wherein the pump includes a pump body having hollow tip portion at a first end and an intake portion at the second end.

4. The method of administration of claim 1, wherein the fluid insert is airless.

5. The method of administration of claim 1, wherein the fluid material is a therapeutic substance.

6. A method of administration comprising:
providing a dispensing apparatus comprising:
an outer casing defining a distal end having an applicator forming a central aperture and a proximal end forming a first opening in communication with a first channel defined along a length of the outer casing;
a fluid insert disposed with the channel, the fluid insert defining a distal end in fluid flow communication with the central aperture and a proximal end defining a second opening in communication with a second channel configured to store a predetermined amount of fluid material therein; and
a sealing plug configured to engage the second opening and seal the second channel;
actuating the dispensing apparatus;
dispensing the fluid material through the applicator for transdermal delivery.

7. The method of administration of claim 6, further comprising a pump disposed within the distal end of the fluid insert for expelling the fluid material through the central aperture.

8. The method of administration of claim 7, wherein the pump includes a pump body having hollow tip portion at a first end and an intake portion at the second end.

9. The method of administration of claim 6, wherein the fluid insert is airless.

10. The method of administration of claim 6, wherein the fluid material is a therapeutic substance.

11. A method of administration comprising:
providing a dispensing apparatus comprising:
an outer casing defining a distal end having an applicator forming a central aperture and a proximal end forming a first opening in communication with a first channel defined along a length of the outer casing;
a fluid insert disposed with the channel, the fluid insert defining a distal end in fluid flow communication with the central aperture and a proximal end defining a second opening in communication with a second channel configured to store a predetermined amount of fluid material therein; and
a sealing plug configured to engage the second opening and seal the second channel;
actuating the dispensing apparatus; and
dispensing the fluid material through the applicator for transmucosal delivery to an individual.

12. The method of administration of claim 11, further comprising a pump disposed within the distal end of the fluid insert for expelling the fluid material through the central aperture.

13. The method of administration of claim 12, wherein the pump includes a pump body having hollow tip portion at a first end and an intake portion at the second end.

14. The method of administration of claim 11, wherein the fluid insert is airless.

15. The method of administration of claim 11, wherein the fluid material is a therapeutic substance.

16. A dispensing apparatus for administration of a fluid material comprising:
an outer casing defining a distal end having an applicator forming a central aperture and a proximal end forming a first opening in communication with a first channel defined along a length of the outer casing;
a fluid insert disposed with the channel, the fluid insert defining a distal end in fluid flow communication with the central aperture and a proximal end defining a second opening in communication with a second channel configured to store a predetermined amount of fluid material therein, wherein the channel is airless; and
a sealing plug configured to engage the second opening and seal the second channel.

17. The dispensing apparatus of claim 16, wherein the fluid material is selected from a group consisting of: a pharmaceutical, a vaccine, a hormone, a large molecule, a small molecule and a nanoparticle.

18. The dispensing apparatus of claim 16, wherein the dispensing apparatus is used to vaginally or labial delivery a therapeutic substance.

19. The dispensing apparatus of claim 16, wherein the dispensing apparatus is used to transmucosal delivery of a therapeutic substance.

20. The dispensing apparatus of claim 16, wherein the dispensing apparatus is used for transdermal delivery of a therapeutic substance.
